(19) United States
(12) Patent Application Publication
(10) Pub. No.: US 2026/0013872 A1
(43) Pub. Date: Jan. 15, 2026

(54) PROXIMAL BEARING FOR SURGICAL BUR FOR USE WITH HIGH SPEED DRILLS

(71) Applicant: Medtronic Xomed LLC, Jacksonville, FL (US)

(72) Inventors: Thoai D. Nguyen, Jacksonville, FL (US); Juliana Neziraj, Jacksonville, FL (US)

(21) Appl. No.: 19/258,083

(22) Filed: Jul. 2, 2025

Related U.S. Application Data
(60) Provisional application No. 63/670,314, filed on Jul. 12, 2024.

(51) Int. Cl. A61B 17/16

(52) U.S. Cl. CPC A61B 17/1633; A61B 17/1628; A61B 17/1631

(57) ABSTRACT

A surgical instrument includes a surgical handpiece having a hub configured to receive a working member therethrough. The working member includes an outer shaft configured to receive a middle tube therethrough and an inner tube concentrically disposed within the middle tube and adapted to connect to a motor 95 at a proximal end thereof. The inner tube is configured to support a surgical working tip at a distal end thereof. The motor 95 is configured to rotate or oscillate the inner tube within the middle tube upon actuation of the motor 95. A bearing is operably disposed within the hub and is configured to support the inner tube during rotation thereof, and to offload both axial forces from the motor 95 and radial forces emanating proximally along the inner tube from the surgical working tip.

### CROSS-REFERENCE TO RELATED APPLICATION

This application claims the benefit of and priority to U.S. Provisional Patent Application No. 63/670,314 filed Jul. 12, 2024, the entire disclosure of which is incorporated by reference herein.

### FIELD

The present disclosure relates generally to high-speed drills for use in surgical procedures, and, more particularly to a proximal bearing for a high-speed drill which supports a surgical bur to reduce vibration of the surgical bur at high rotational speeds.

### BACKGROUND

Surgical procedures can be performed on anatomies such as the human anatomy for providing a therapy to the anatomy. One area of surgery includes procedures performed on facial cavities of a patient such as on the car, nose or throat (ENT). In such procedures, a surgical cutting tool such as a shaver, bur, or other tool connected to an instrument (e.g., a handpiece) may be inserted into such a cavity to cut tissue, remove bone, or perform a bone-shaping procedure. During use of the surgical instrument, it is favorable to avoid trauma to other sensitive areas, unintentionally make any supplemental incisions, and avoid other axis portals in the patient tissue.

Increasing the rotational speed of the surgical bur (or surgical cutting tool) increases the effectiveness of the tissue and bone removal process. Current attempts to provide higher speed drills, e.g., drills rotating above 30,000 RPM, have been challenging due to vibrational issues within the instrument and with the shaft.

As such, there is a need to provide reliable and inexpensive way to increase the rotational speed of the cutting tool, while avoiding the drawbacks associated with the same.

### SUMMARY

Provided in accordance with aspects of the present disclosure is a surgical instrument which includes a surgical handpiece having a hub configured to receive a working member therethrough. The working member includes an outer shaft configured to receive a middle tube therethrough and an inner tube concentrically disposed within the middle tube and adapted to connect to a motor at a proximal end thereof. The inner tube is configured to support a surgical working tip at a distal end thereof. The motor is configured to rotate or oscillate the inner tube within the middle tube upon actuation of the motor. A bearing is operably disposed within the hub and is configured to support the inner tube during rotation thereof, the bearing configured to offload both axial forces from the motor and radial forces emanating proximally along the inner tube from the surgical working tip.

In aspects according to the present disclosure, the surgical instrument further includes a stub member operably disposed between the motor and the hub atop the inner tube, the stub member configured to transfer the axial load from the motor to the bearing. In other aspects according to the present disclosure, the motor is disposed in a tapered hub and includes a compression spring which is configured to operably couple to the stub member.

In aspects according to the present disclosure, the surgical working tip is supported by a second bearing for rotation within the middle tube.

In aspects according to the present disclosure, the bearing includes a plurality of ball bearings, the plurality of ball bearings configured to offload the radial forces emanating proximally along the inner tube from the surgical working tip during rotation thereof.

In aspects according to the present disclosure, the second bearing includes a plurality of second ball bearings, the plurality of second ball bearings configured to seat within a raceway defined within the surgical working tip, the plurality of second ball bearings configured to project radially through a corresponding plurality of holes defined in the middle tube to abut against the outer shaft, and to offload radial forces from the surgical working tip during rotation thereof against the outer shaft.

In aspects according to the present disclosure, the bearing is at least one of press-fit, screwed, snap-fit, or welded within the hub.

In aspects according to the present disclosure, the inner tube includes a step defined therein, the step configured to receive the bearing thereon, and to prevent lateral movement of the bearing atop the inner tube.

In aspects according to the present disclosure, the stub member is welded atop the inner tube between the compression spring of the motor and the hub.

In aspects according to the present disclosure, the inner tube is flexible.

Provided in accordance with aspects of the present disclosure is a surgical instrument which includes a surgical handpiece and a first hub configured to support a motor therein, the motor including a compression ring. A second hub is configured to receive a working member therethrough, the working member including: an outer shaft configured to receive a middle tube therethrough; and an inner tube concentrically disposed within the middle tube and adapted to connect to the motor at a proximal end thereof, the inner tube configured to support a surgical working tip at a distal end thereof. The motor is configured to at least one of rotate or oscillate the inner tube within the middle tube upon actuation of the motor, which, in turn, rotates or oscillates the surgical working tip. A bearing is operably disposed within the second hub and configured to support the inner tube during rotation thereof, and to offload both axial forces from the compression spring of the motor and radial forces emanating proximally along the inner tube from the surgical working tip. A stub member is operably disposed between the compression spring of the motor and the second hub, the stub member operably engaged atop the inner tube and configured to transfer the axial load from the compression spring of the motor to the bearing.

In aspects according to the present disclosure, the first hub is tapered.

In aspects according to the present disclosure, the surgical working tip is supported by a second bearing for rotation within the middle tube.

In aspects according to the present disclosure, the bearing includes a plurality of ball bearings configured to offload the radial forces emanating proximally along the inner tube from the surgical working tip during rotation thereof.

In aspects according to the present disclosure, the second bearing includes a plurality of second ball bearings, the plurality of second ball bearings configured to seat within a raceway defined within the surgical working tip, the plurality of second ball bearings configured to project radially through a corresponding plurality of holes defined in the middle tube to abut against the outer shaft, and to offload radial forces from the surgical working tip during rotation thereof against the outer shaft.

In aspects according to the present disclosure, the bearing is at least one of press-fit, screwed, snap-fit, or welded within the second hub.

In aspects according to the present disclosure, the inner tube includes a step defined therein, the step configured to receive the bearing thereon, and to prevent lateral movement of the bearing atop the inner tube.

In aspects according to the present disclosure, the stub member is welded atop the inner tube between the compression spring of the motor and the second hub.

In aspects according to the present disclosure, the inner tube includes a flexible portion.

### BRIEF DESCRIPTION OF THE DRAWINGS

The drawings described herein are for illustration purposes only and are not intended to limit the scope of the present disclosure in any way.

FIG. 1 is an environmental view of a prior art surgical navigation system, according to various embodiments;

FIG. 2 is a perspective view of a prior art handpiece having a surgical bur disposed at a distal end of an elongated shaft of the surgical instrument;

FIG. 3 is a perspective view of the handpiece of FIG. 2 with a proximal end shown transparent;

FIG. 4 is an enlarged, side cross-section of a proximal end of the handpiece of FIG. 2 showing the electromagnetic tracking device mounted thereon;

FIG. 5 is an internal, side cross-section of the handpiece of FIG. 2 highlighting a series of tracking coils disposed within the handpiece;

FIG. 6A is an internal cross-section of the handpiece of FIG. 2;

FIG. 6B is a greatly enlarged internal cross-section of a distal end of the surgical instrument of FIG. 2;

FIGS. 6C-6E are greatly-enlarged, perspective views of the surgical bur being operably engaged with a distal end of the shaft of the handpiece;

FIGS. 7A-7D are various views showing the various forces associated with the handpiece of FIG. 2 during rotation of the surgical bur;

FIG. 8A is an internal cross-section of a handpiece in accordance with the present disclosure including a surgical bur operably engaged to an internally disposed rotating inner tube;

FIG. 8B is a greatly-enlarged, internal cross-section of a proximal end of the handpiece showing a bearing configured to support the inner rotating tube;

FIG. 8C is a greatly-enlarged, internal cross-section of a distal end of the surgical instrument of FIG. 8A; and

FIG. 8D is a schematic diagram showing the various forces associated with a second bearing disposed proximate the surgical bur.

### DETAILED DESCRIPTION

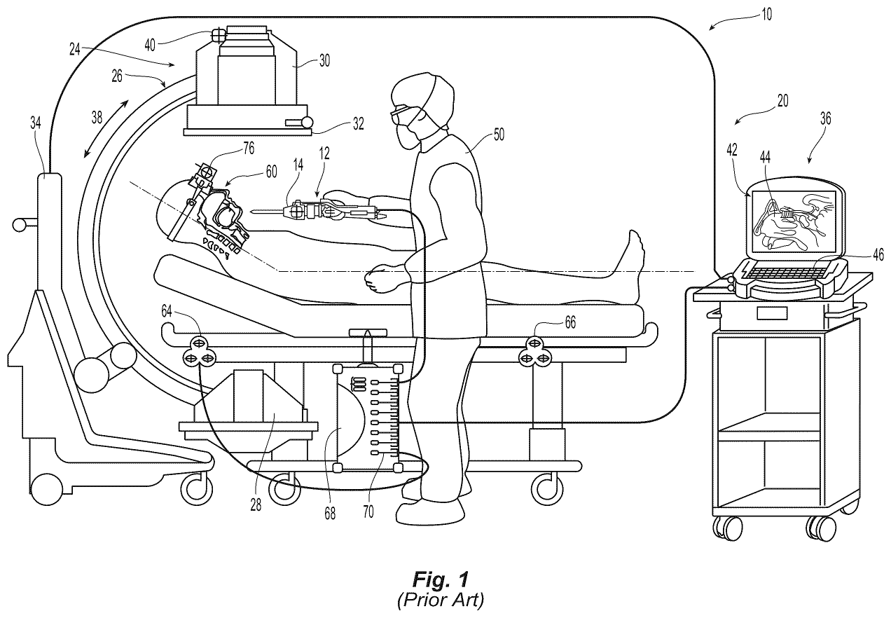

With initial reference to FIG. 1, one example of an electromagnetic (EM) image-guided surgery system according to one example of the present teachings is shown and generally identified as reference numeral 10. The EM image-guided surgery system 10 can generally include an instrument such as a handpiece 12 having an EM instrument tracking device 14 configured to communicate with an exemplary navigation system 20. The navigation system 20 can be used to track the location of the handpiece 12 relative to a patient 22 to assist in a surgical procedure.

As will be described in greater detail herein, the EM instrument tracking device 14 can receive a signal, transmit a signal, or combinations thereof to provide information to the navigation system 20 to determine a location of the handpiece 12. Prior to discussing in detail the EM instrument navigational system, a general discussion of the prior art exemplary navigation system 20 is warranted.

The navigation system 20 includes an imaging device 24 that is used to acquire pre-, intra-, or post-operative or real-time image data of the patient 22. The image data acquired with the imaging device 24 can be used as part of the image data in the EM image-guided surgery system 10. In addition, data from atlas models can be used to produce patient images. The imaging device 24 may be a fluoroscopic X-ray imaging device with a C-arm 26 having an X-ray source 28, an X-ray receiving section 30, an optional calibration and tracking target 32 and optional radiation sensors.

The EM instrument tracking device 14 is coupled to the handpiece 12 such that the navigation system 20 can track the location of the handpiece 12 relative to a patient 22 to assist in surgical procedure. Image data is forwarded from the imaging device 24 to the navigation computer and/or processor controller or work station 36 having a display device 42 to display image data 44 and a user interface 46. Processing for the navigation system 20 and optimization can all be done with a single or multiple processors all of which may or may not be included in the work station 36.

The work station 36 provides facilities for displaying the image data 44 as an image on the display device 42, saving, digitally manipulating, and/or printing a hard copy image of the received image data. The user interface 46, which may be a keyboard, mouse, touch pen, touch screen or other suitable device, allows a physician or user 50 to provide inputs to control the imaging device 24, via the controller 34, or adjust the display settings of the display 42.

Although one type of imaging device 24 is shown in FIG. 1, any other alternative 2D, 3D or 4D imaging modality may also be used. The images may also be obtained and displayed in two, three or four dimensions.

The tracking devices 14, 40, 76 or any tracking device as discussed herein, can include a sensor, a transmitter, or combinations thereof. Further, the tracking devices can be wired or wireless to provide a signal emitter or receiver within the navigation system 20. For example, any or all of the tracking devices 14, 40, 76 can include an electromagnetic coil to sense a field produced by the localizing array 64, 66. The tracking device (14, 40, 76) may be configured to receive a signal, transmit a signal, or combinations thereof to provide information to the navigation system 20 to determine a location of the tracking device 14, 40, 76. Therefore, as used herein, “generating an output signal” is used to mean any combination of receiving a signal, transmitting a signal or combinations thereof. The navigation system 20 can then determine a position of the handpiece 12 or tracking device 14, 40, 76 to allow for navigation relative to the patient 22 and patient space.

The EM image-guided surgery system 10 uses the coil arrays 64, 66 to create an electromagnetic field used for navigation. The coil array 64 is controlled or driven by the coil array controller 68. The coil array controller 68 drives each coil in the coil array 64 in a time division multiplex or a frequency division multiplex manner.

Upon driving the coils in the coil array 64 with the coil array controller 68, electromagnetic fields are generated within the patient 22 in the area where the medical procedure is being performed, which is sometimes referred to as patient space. The electromagnetic fields generated in the patient space induce currents in the EM instrument tracking device 14 and patient tracking device 76. These induced signals from the tracking devices 14, 40, 76 are delivered to the navigation handpiece interface 70 and subsequently forwarded to the coil array controller 68.

Briefly, the navigation system 20 operates as follows. The navigation system 20 creates a translation map between all points in the image data generated from the imaging device 24 which can include external and internal portions, and the corresponding points in the patient's anatomy in patient space. After this map is established, whenever the tracked handpiece 12 is used, the work station 36 in combination with the coil array controller 68 uses the translation map to identify the corresponding point on the image data or atlas model, which is displayed on display 42. This identification is known as navigation or localization. An icon representing the localized point or instruments is shown on the display 42 within several two-dimensional image planes, as well as on three and four dimensional images and models.

To enable navigation, the navigation system 20 must be able to detect both the position of the patient's anatomy and the position of the handpiece 12 (or EM instrument tracking device 14) attached to the handpiece 12. Knowing the location of these two items allows the navigation system 20 to compute and display the position of the handpiece 12 or any portion thereof (e.g., a working member as will be described) in relation to the patient 22. The EM image-guided surgery system 10 is employed to track the handpiece 12 and the anatomy simultaneously.

The EM image-guided surgery system 10 essentially works by positioning the coil array 64, 66 adjacent to the patient 22 to generate a magnetic field, which can be low energy, and generally referred to as a navigation field. Because every point in the navigation field or patient space is associated with a unique field strength, the EM image-guided surgery system 10 can determine the position of the handpiece 12 by measuring the field strength at the location of the EM instrument tracking device 14. The dynamic reference frame 60 is fixed to the patient 22 to identify the location of the patient 22 in the navigation field. The EM image-guided surgery system 10 continuously recomputes or recalculates the relative position of the dynamic reference frame 60 and the handpiece 12 during localization and relates this spatial information to patient registration data to enable navigation of the handpiece 12 within and/or relative to the patient 22. Navigation can include image guidance or imageless guidance.

The EM image-guided surgery system 10 may also perform 2D to 3D registration by utilizing the acquired 2D images to register 3D volume images by use of contour algorithms, point algorithms or density comparison algorithms, as is known in the art. An exemplary 2D to 3D registration procedure is set forth in U.S. Pat. No. 7,570,791, hereby incorporated by reference.

In order to maintain registration accuracy, the navigation system 20 continuously tracks the position of the patient 22 during registration and navigation. This is because the patient 22, dynamic reference frame 60, and transmitter coil array 64, 66 may all move during the procedure, even when this movement is not desired. Therefore, if the navigation system 20 did not track the position of the patient 22 or area of the anatomy, any patient movement after registration would result in inaccurate navigation within that image. The dynamic reference frame 60 allows the EM image-guided surgery system 10 to register and track the anatomy. Because the dynamic reference frame 60 is rigidly fixed to the patient 22, any movement of the anatomy or the coil array 64, 66 is detected as the relative motion between the coil array 64, 66 and the dynamic reference frame 60. This relative motion is communicated to the coil array controller 68, via the navigation handpiece interface 70, which updates the registration correlation to thereby maintain accurate navigation.

Generally, the navigation system 20 allows the images on the display 42 to be registered and accurately display the real time location of the various instruments and other appropriate items. In addition, the handpiece 12 may be used to register the patient space to the pre-acquired image data or the atlas or 3D models. In addition, the dynamic reference frame 60 may be used to ensure that any planned or unplanned movement of the patient or the array 64, 66 is determined and used to correct the image on the display 42.

As mentioned briefly above, the display 42 can display any appropriate type of image data 44. For example, the image data 44 can include patient specific image data that can be acquired at any appropriate time. The image data can include magnetic resonance imaging data (MRI) that can provide structural anatomical image data (such as a nasal cavity, etc.) of the patient 22. The image data 44 can be displayed on the display 42 for use during a procedure by the user 50.

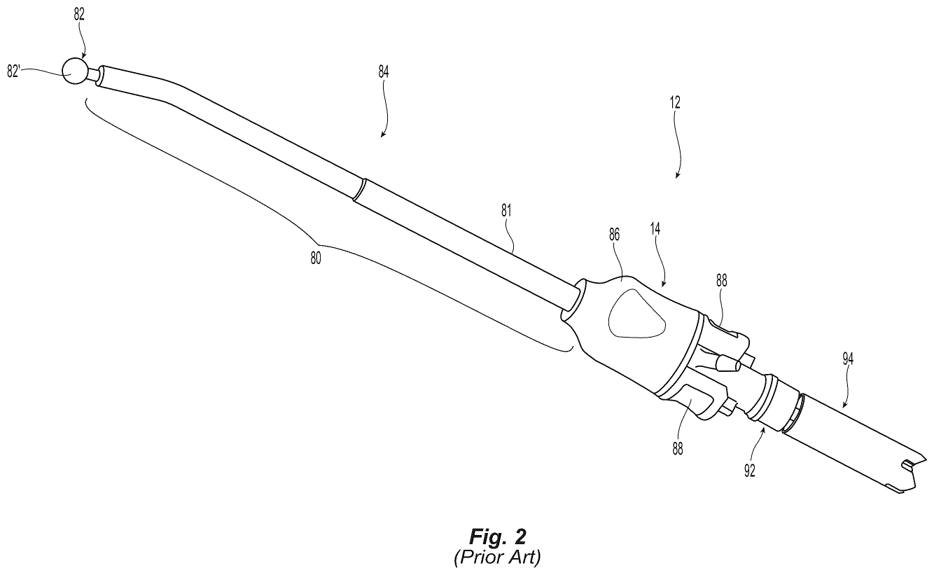

With reference now to FIG. 2, the electromagnetic tracking device 14 is shown operatively coupled with the example prior art handpiece 12. The handpiece 12 may include an attached working member 80 having a surgical working tip 82 (e.g., a surgical bur or surgical cutting blade). The working member 80 may include a surgical working tip 82 specifically adapted for car, nose and throat (ENT) surgical procedures for burring or shaping various ENT cavities. While only one working member 80 is shown, the EM image-guided surgery system 10 can include a plurality of working members 80 each having different characteristics. The location of a respective surgical working tip 82 for each working member 80 can be programmed into the image-guided surgery system 10, such as at a terminal connector of the handpiece 12.

In one example, the working member 80 can be part of a blade assembly 84, FIGS. 2 and 3 that is fixed to the electromagnetic tracking device 14. In another example, the working member 80 or the blade assembly 84 as a whole can be releasably coupled to the electromagnetic tracking device 14. The blade assembly 84 can include an outer hub 86 having connecting tabs 88 for releasably attaching the outer hub 86 atop an inner hub 92. Inner hub 92 can be arranged intermediate of a tapered hub 94 and the outer hub 86. In an assembled position (FIG. 2), the connecting mechanism 88 can engage structure (not specifically shown) on the inner hub 92. The inner hub 92 may include an irrigation connector 90 which is configured for attachment to an aspiration unit (not shown). On assembly, the distal end of the handpiece 12 is inserted through a passthrough defined by the base opening 126.

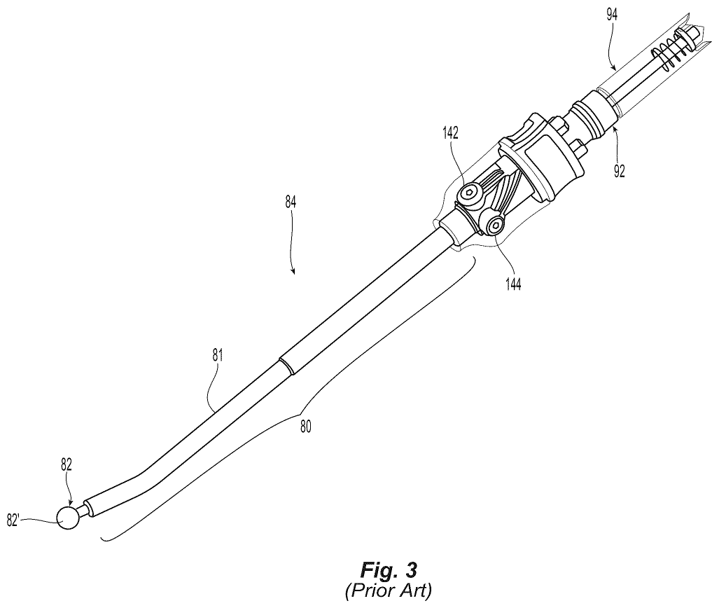

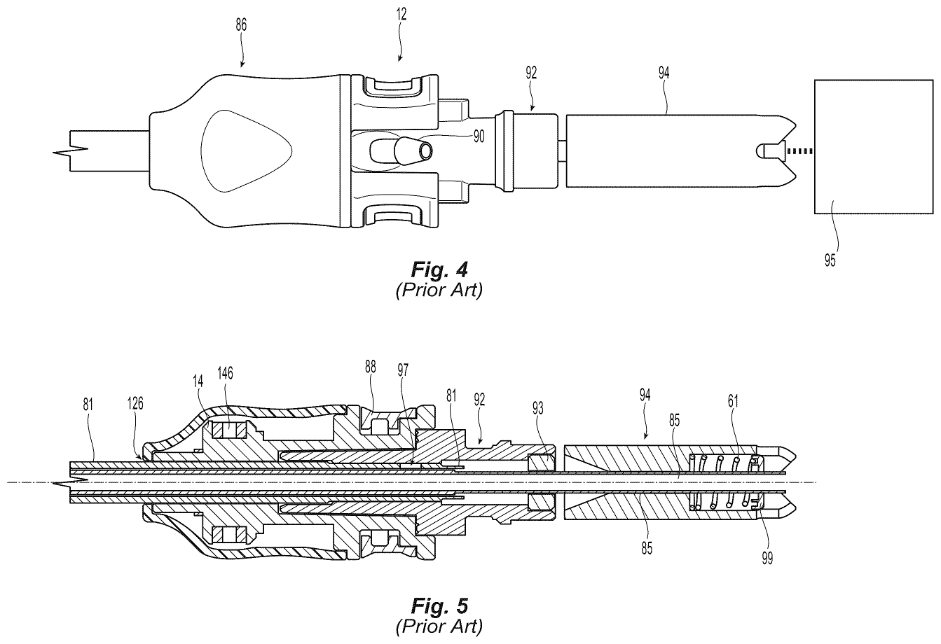

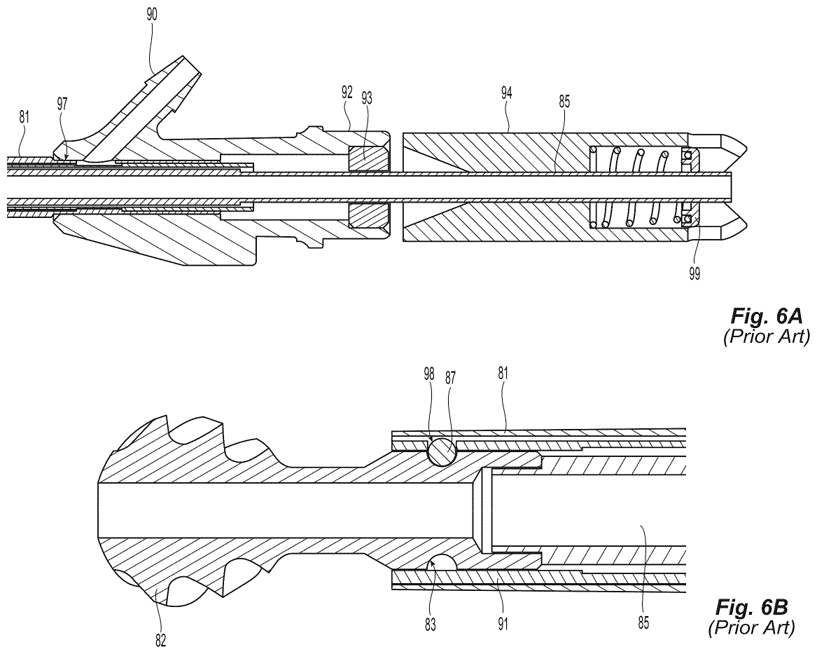

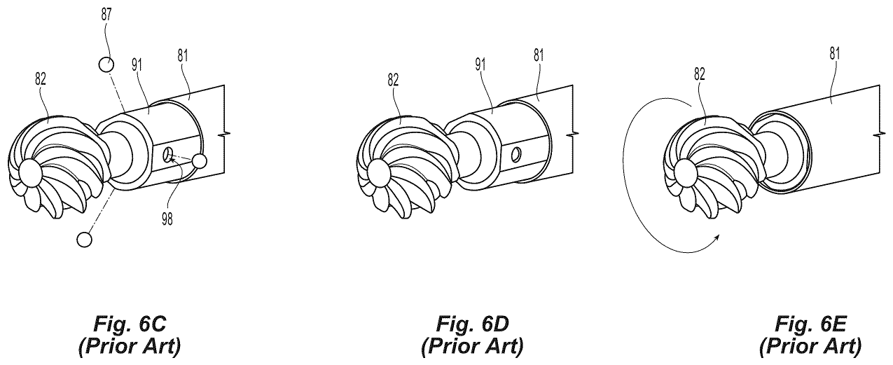

With reference to FIGS. 4 and 5, during use, a motor 95 rotates an inner tube 85 relative to the outer hub 86. Inner tube 85, extends through outer shaft 81 of the working member 80 and supports the surgical working tip 82 at a distal end thereof. As shown in the prior art embodiment of FIG. 5, a bushing 93 supports the inner tube 85 at a proximal end thereof between the inner hub 92 and the tapered hub 94. Motor 95 operably couples to the tapered hub 94 and a proximal-most portion of the inner tube 85 and, upon activation, rotates the inner tube 85 within a middle tube 91 (FIG. 6B). In turn, rotation of the inner tube 85 is imbued onto the surgical working tip 82 upon activation of the motor 95 by the surgeon. Fluid may be supplied to the surgical working tip 82 via one or more irrigation ports 97 defined in the outer shaft 81 (FIGS. 5 and 6A) which register with the irrigation connector 90 upon assembly of the handpiece 12.

FIGS. 6A-6E show an internal side view of the prior art surgical handpiece 12 which includes a bushing 93 disposed within inner hub 92. Bushing 93 is configured to support the inner tube 85 and prevent the inner tube 85 from touching the middle tube 91 during rotation.

At the distal end of the surgical handpiece 12, the surgical working tip 82 is securely coupled to the inner tube 85 via some form of mechanical connection. As best shown in FIGS. 6C-6E, a set of ball bearings 87 is partially seated within a raceway 83 defined in a proximal end of the surgical working tip 82 and corresponding holes 98 defined in the middle tube 91. During rotation of the inner tube 85 within the middle tube 91, the ball bearings 87 support the rotation at the distal end and constrain the inner tube 85 from moving axially.

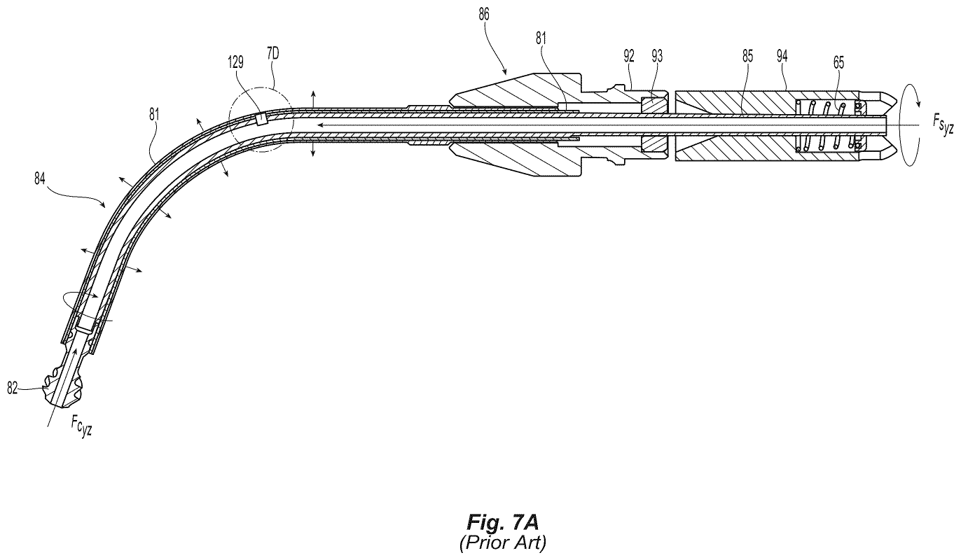

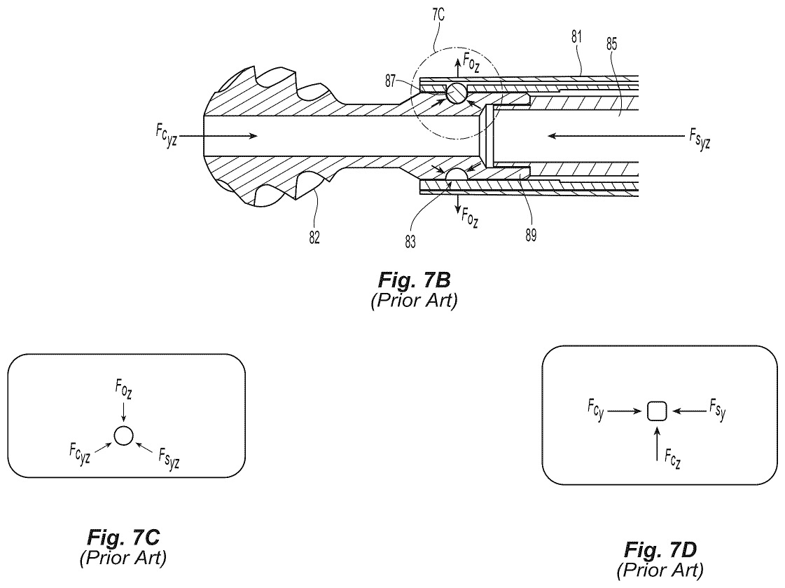

At lower rotational speeds, the forces associated with the inner rotating tube are minimized by the ball bearings 87 and the proximal bushing 93. More particularly, and as best shown in FIGS. 7A-7D, the cutting force Fc_{yz} associated with the surgical working tip 82 are offloaded by the ball bearings 87 at the distal end of the handpiece 12. More particularly, and as shown in FIGS. 7A and 7C, most of the forces, cutting force (Fc_{yz}), axial spring force from hub spring 61 disposed in hub 94 (Fs_{yz}), and the force associated with the outer shaft 81 on the ball bearings 87 (Fo_{z}). Moreover, along the bend of the inner tube 85, the associated forces, namely, the cutting forces Fc_{y} and Fc_{z} and the opposing spring force Fs_{y} are also offloaded by a combination of the bushing 93 and bearings 87 (FIG. 7D).

At higher drilling speeds, e.g., above 30K RPM, there is a tendency for the cutting forces associated with the surgical working tip 82, namely cutting forces Fc_{y} and Fc_{z} and the opposing spring force Fs_{y} along the inner tube 85 to become imbalanced causing radial forces/vibration and creating one or more friction points 129 between the inner tube 85 and the middle tube 91. Typically this is where the opposing forces (cutting forces Fc_{y} and Fc_{z} and the opposing spring force Fs_{y}) meet along the inner tube 85.

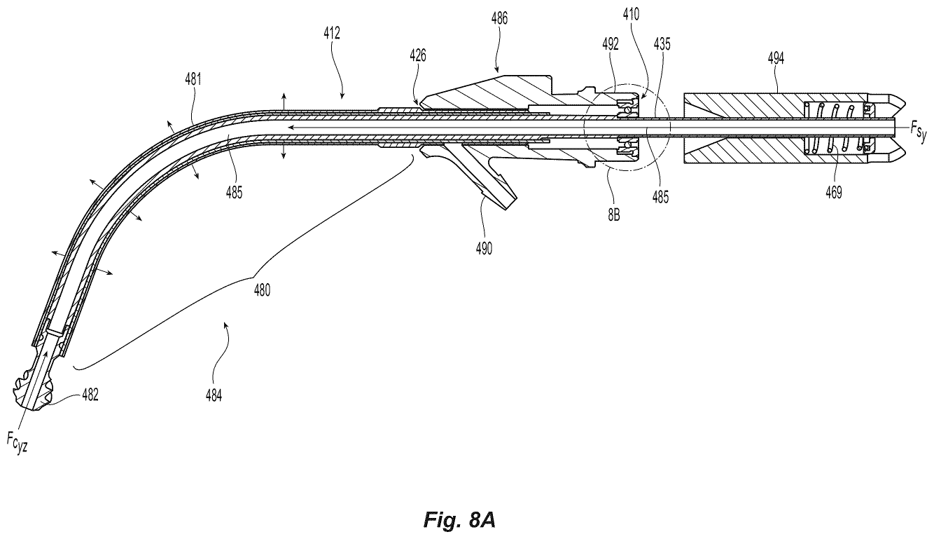

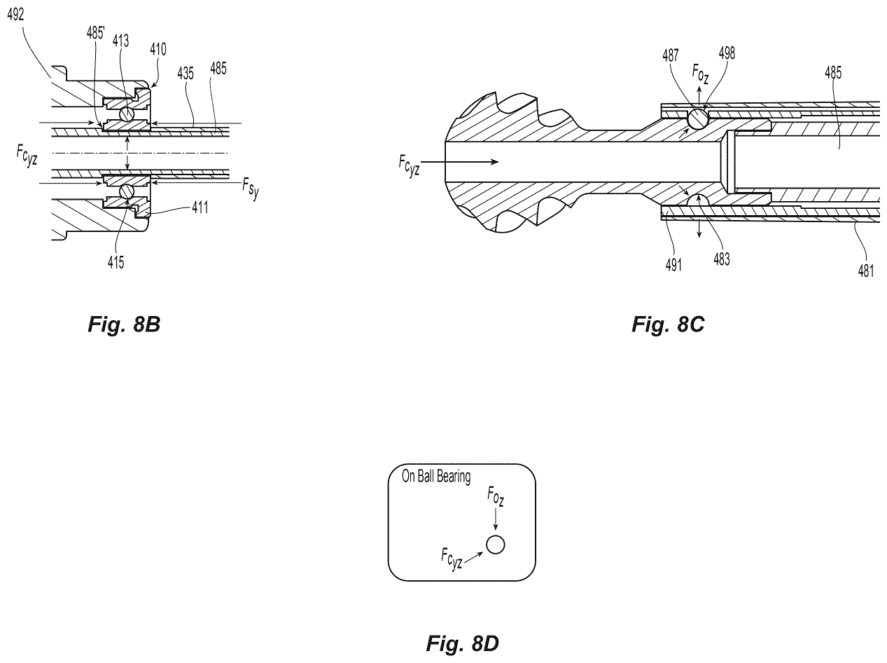

The present disclosure provides a solution for the present imbalance between the cutting forces Fc_{y} and Fc_{z} and the opposing spring force Fs_{y} and allows an inner tube 485 to rotate at much higher speeds with little or no radial forces/vibration due to unwanted friction between the inner tube 485 and middle tube 491. As shown in the embodiment of FIGS. 8A-8C, a surgical handpiece 412 includes many of the same above-described components and for the purposes of brevity are reiterated herein only as necessary to describe the novel features associated with handpiece 412.

FIG. 8A shows an internal cross-section of the presently disclosed handpiece 412 which includes a removably (or non-removably) attached working member 480 having a suitable surgical working tip 482 (e.g., a surgical bur or surgical blade tip).

Working member 480 is part of a bur assembly 484 that is fixed to the outer hub 486 via connection tabs which releasably couple to the inner hub 492 similar to connection tabs 88 described above. The inner hub 492 may include an irrigation connector 490 which is configured for attachment to an aspiration unit (not explicitly shown). On assembly, the distal end of the handpiece 412 is inserted through a passthrough defined by the base opening 426.

During use, a motor (not explicitly shown in FIG. 8A but similar to motor 95 of FIG. 4) rotates inner tube 485 relative to the outer hub 486. Inner tube 485 extends through outer shaft 481 of the working member 480 and supports the surgical working tip 482 at a distal end thereof. A bearing 410 supports the inner tube 485 at a proximal end of hub 492 (e.g., between the inner hub 492 and the tapered hub 494). Motor operably couples to the tapered hub 494 and a proximal-most portion of the inner tube 485 and, upon activation, rotates or oscillates (or both) the inner tube 485 within middle tube 491. In turn, rotation of the inner tube 485 is imbued onto the surgical working tip 482 upon activation of the motor by the surgeon. Fluid may be supplied to the surgical working tip 482 via one or more irrigation ports (similar to ports 97 in FIG. 2 above) defined in the outer shaft 481 which register with the irrigation connector 490 upon assembly of the handpiece 412.

FIG. 8B shows an enlarged, internal cross-section of the bearing 410 disposed within the proximal end of inner hub 492. Bearing 410 may be seated, press-fit, snap-fit, screwed or welded within inner hub 492. Inner tube 485 includes a step or notched portion 485′ configured to operably engage and support the bearing 410 atop the inner tube 485 and prevent lateral movement of the bearing 410 along the inner tube 485. Bearing 410 also includes a bushing outer flange 411 which is configured to operably engage a proximal end of the inner hub 492 and facilitate securing bearing 410 therein.

Bearing 410 includes a plurality of ball bearings 413 that are configured to offload the cutting forces (Fc_{y,} and/or Fc_{z}) proximally emanating along the inner tube 485 and the spring forces (Fs_{y}) from the tapered hub 494. More particularly, any vibrations (e.g., unbalanced radial and/or axial loads) emanating proximally along the inner tube 485 are offloaded by the ball bearings 413 at bearing 410 and any forces or vibrations (unbalanced radial and/or axial loads) emanating distally along the inner 485 tube from the tapered hub 494 are offloaded at the bearing 410. A stub or tubular cuff 435 may be disposed atop the inner tube 485 between inner hub 492 and tapered hub 494. Stub 435 is configured to transfer the axial load from the compression spring 469 associated with the motor to the bearing 410. The stub 435 is configured to absorb torque from the tapered hub 494 and transmits the torque to the inner tube 485. In turn, the stub 435 pushes against the bearing 410 to ensure no axial forces, e.g., an axial compressive force, is applied along the inner tube 485 and potentially to any flexible or articulating portion thereof. Stub 435 may be welded to an outer periphery of the inner tube 485 between the inner hub 492 and the tapered hub 494.

Similar to the above-described embodiment of FIGS. 7B-7D, the cutting forces Fc_{yz} and Fo_{z} associated with the surgical working tip 482 are offloaded by the ball bearings 487 at the distal end of the handpiece 412. However, since spring forces Fs_{y} are offloaded at the proximal end of the inner tube 485 by bearing 410 (FIG. 8C), a majority of the cutting forces Fc_{yz} are offloaded by bearings 487 and are not transferred on the inner tube 485, thereby resulting in reduced radial forces/vibration therealong.

While several aspects of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope of the claims appended hereto.

What is claimed is:

1. A surgical instrument, comprising:
a surgical handpiece including a hub configured to receive a working member therethrough, the working member including:
an outer shaft configured to receive a middle tube therethrough; and
an inner tube concentrically disposed within the middle tube and adapted to connect to a motor at a proximal end thereof, the inner tube configured to support a surgical working tip at a distal end thereof, the motor configured to at least one of rotate or oscillate the inner tube within the middle tube upon actuation of the motor; and
a bearing disposed within the hub and configured to support the inner tube during rotation thereof, the bearing configured to offload both axial forces from the motor and radial forces emanating proximally along the inner tube from the surgical working tip.

2. The surgical instrument according to claim 1, further comprising a stub member operably disposed between the motor and the hub atop the inner tube, the stub member configured to transfer the axial load from the motor to the bearing.

3. The surgical instrument according to claim 2, wherein the motor is disposed in a tapered hub and includes a compression spring that is configured to operably couple to the stub member.

4. The surgical instrument according to claim 1, wherein the surgical working tip is supported by a second bearing for rotation within the middle tube.

5. The surgical instrument according to claim 1, wherein the bearing includes a plurality of ball bearings, the plurality of ball bearings configured to offload the radial forces emanating proximally along the inner tube from the surgical working tip during rotation thereof.

6. The surgical instrument according to claim 4, wherein the second bearing includes a plurality of second ball bearings, the plurality of second ball bearing configured to seat within a raceway defined within the surgical working tip, the plurality of second ball bearings configured to project radially through a corresponding plurality of holes defined in the middle tube to abut against the outer shaft, the plurality of second bearings configured to offload radial forces from the surgical working tip during rotation thereof against the outer shaft.

7. The surgical instrument according to claim 1, wherein the bearing is at least one of press-fit, screwed, snap-fit, or welded within the hub.

8. The surgical instrument according to claim 1, wherein the inner tube includes a step defined therein, the step configured to receive the bearing thereon, the step configured to prevent lateral movement of the bearing atop the inner tube.

9. The surgical instrument according to claim 3, wherein the stub member is welded atop the inner tube between the compression spring of the motor and the hub.

10. The surgical instrument according to claim 1, wherein the inner tube is flexible.

11. A surgical instrument, comprising:
a surgical handpiece;
a first hub configured to operably support a motor therein, the motor including a compression spring;
a second hub configured to receive a working member therethrough, the working member including:
an outer shaft configured to receive a middle tube therethrough; and
an inner tube concentrically disposed within the middle tube and adapted to connect to the motor at a proximal end thereof, the inner tube configured to support a surgical working tip at a distal end thereof, the motor configured to at least one of rotate or oscillate the inner tube within the middle tube upon actuation of the motor, which, in turn, rotates or oscillates the surgical working tip;
a bearing operably disposed within the second hub and configured to support the inner tube during rotation thereof, the bearing configured to offload both axial forces from the compression spring of the motor and radial forces emanating proximally along the inner tube from the surgical working tip; and
a stub member operably disposed between the compression spring of the motor and the second hub, the stub member operably engaged atop the inner tube and configured to transfer the axial load from the compression spring of the motor to the bearing.

12. The surgical instrument according to claim 11, wherein the first hub is tapered.

13. The surgical instrument according to claim 11, wherein the surgical working tip is supported by a second bearing for rotation within the middle tube.

14. The surgical instrument according to claim 11, wherein the bearing includes a plurality of ball bearings, the plurality of ball bearings configured to offload the radial forces emanating proximally along the inner tube from the surgical working tip during rotation thereof.

15. The surgical instrument according to claim 13, wherein the second bearing includes a plurality of second ball bearings, the plurality of second ball bearings configured to seat within a raceway defined within the surgical working tip, the plurality of second ball bearings configured to project radially through a corresponding plurality of holes defined in the middle tube to abut against the outer shaft, the plurality of second ball bearings configured to offload radial forces from the surgical working tip during rotation thereof against the outer shaft.

16. The surgical instrument according to claim 11, wherein the bearing is at least one of press-fit, screwed, snap-fit, or welded within the second hub.

17. The surgical instrument according to claim 11, wherein the inner tube includes a step defined therein, the step configured to receive the bearing thereon, the step configured to prevent lateral movement of the bearing atop the inner tube.

18. The surgical instrument according to claim 11, wherein the stub member is welded atop the inner tube between the compression spring of the motor and the second hub.

19. The surgical instrument according to claim 11, wherein the inner tube includes a flexible portion.

20. The surgical instrument according to claim 11, wherein the surgical working tip is at least one of a debrider, surgical bur, or surgical blade.
